(19) United States
(12) Patent Application Publication
(10) Pub. No.: US 2002/0143280 A1
(43) Pub. Date: Oct. 3, 2002

(54) Best multifunction bucks traction boot

(76) Inventors: Sean J. Souney, Monrovia, CA (US); Richard W. Roeder, Monrovia, CA (US); Kurt Fuhrman, Thousand Oaks, CA (US)

Correspondence Address: Sean Souney, 730 East Los Angeles Ave., Monrovia, CA, 91016, US

(21) Appl. No.: 10/044,417

(22) Filed: Jan. 10, 2002

Related U.S. Application Data
(60) Provisional application No. 60/260,775, filed on Jan. 10, 2001.

(51) Int. Cl. A61F 5/00

(52) U.S. Cl. 602/32

(57) ABSTRACT

A universal bucks traction boot for orthopedic traction on the leg having an asymmetrical shape which when connected together forms a heel pocket and has attached to it a stirrup of webbing that is reinforced with plastic stays and has a friction free movable circular ring around the webbing for attachment. The convoluted foam inside surface has a hook compatible surface on the outside of the material for the attachment of the wide straps. In addition, a large dorsal area that is cut out allows access to the foot for patient assessment or for the application of an external/intermittent pneumatic compression device that is often needed to be used with the patient who needs orthopedic traction. The foam will be of soft durometer but of sufficient density so that when attempts are made to over tighten the straps the hook and loop interface will release and not allow over tightening.

### BACKGROUND-FIELD OF INVENTION

This invention relates to a multifunction bucks traction boot device used to provide orthopedic traction on the leg after leg trauma or surgery in combination with an intermittent pneumatic compression device used for the prevention of deep vein thrombosis and pulmonary embolism.

1. Background-Description of Prior Art

To provide orthopedic traction, typically a weight is attached to the end of the orthopedic boot to pull on the leg through the centerline of the leg to reduce the lateral and positional stresses from moving the orthopedic set of the bones. This position allows the traumatized leg to be positioned for proper healing.

Prior art reveals boots that in general support the leg by virtue of a harness design. To secure the harness to the leg, it is strapped tight circumferencially from the proximal portion below the knee to the distal portion which wraps around the foot. The tightness of the straps insures that the pull of the weight does not pull the boot off. Often because of the tightness needed to secure the harness from pulling off a loop is placed on the side of the boot to allow the application specialist to gain leverage from pulling to allow the ratcheting of the strap tight before securing to a buckle or hook and loop closure. The tightness of the straps is needed because the lower leg, specifically the calf, tends to be a tapered circumference as one moves from the knee distally. This means that without fairly tight strapping using thin straps to generate friction on the leg, the device runs the risk of falling off the leg when the traction is applied. In fact, in all facets of devices that are designed to hold on to the leg and permit a weight to pull the leg down its centerline suffer from major disadvantages.

(a) Currently, constriction straps are thin (1-1.5 inches) in width and have to be applied tightly and with four or more locations on the lower leg and additionally across the dorsum of the foot which is not only uncomfortable but is additionally constricting on the dorsal veins. To accomplish the tension needed through the use of a thin strap, slide buckles are often used to allow the application technician the leverage needed for tightening and developed through a 180-degree pull with a ratcheting action.

This easily creates a situation where the straps may be over-tightened causing pain, discomfort, and possible circulatory deficits. Additionally, with leverage buckles or web tabs that have hook and closure attachment, a disproportionate leg shape may result in the inability to match up the strap with its attachment point. Furthermore, the leverage buckle system, in particular, is difficult to apply and even more difficult to assess correct tightness to remedy a possible over-tightening. This is particularly important when treating patients with acute injuries on the leg such as proximal hip fractures where ease of application is critical to setting up a successful traction.

b) The two types of attachment points for the traction cord (which is used to attach the weight for the application of traction) are the ‘spreader bar’ and the ‘spreader plate’. The bar design has two points where it is formed into a loop to accept and hold onto traction cord and a singular attachment point for the boot. The problems inherent in this design are that it must be looped at these two points prior to use. The ease of looping the traction cord at these two points of contact has the unfortunate ability to be able to fall off with the same ease with which it was looped with minimal leg movement. Additionally, when not pre-centered to provide a centered pull on the leg (since there are two separate traction cord attachment points to cause friction) there is the tendency for unequal pull on the and/or the chance that a rope may fall off the loop of the spreader bar with minimal leg movement. A refinement of this design followed and is widely used called the spreader plate which through the use of closed slots allows a woven webbing to be attached to the boots after being threaded through the closed slots. This eliminates the possibility of the boot falling out of its attachment points. One of the problems associated with this refinement is the slotting and webbing can become bunched upon leg movement because of the additional friction from the webbing moving in a thin slot. This causes even worse misalignment in the intended straight pull of the device, leaving the plate shifted so equal pull on both sides of the boot (through the centerline of the limb) is not possible unless there is intervention to straighten it.

(c) With defined strap lengths at multiple positions up the leg, and their linked nature affixed to a dimensioned harness, different sizes (small, medium, large) are needed to accommodate the differing leg dimensions from the dorsum of the foot to the end of the belly of the gastrocnemius.

(d) As dorsum straps are typically employed to provide needed securing positions for the pull on the leg (and the toes of the foot are often covered with this employed design), they can often interfere with the ability to inspect the distal leg pulse, tissue integrity and additional neurological checks. In addition to interference, over-tightened dorsum straps may be the cause of health risks in the foot related to pulse, tissue integrity and neurological dysfunction since knowledge of over-tightening is difficult to assess until the detrimental symptoms present themselves.

(e) As most boots are constructed of a similar open cell foam, they have the undesirable component of friction against the bed surface. Desired pulls on the leg may be starting as low as 2.5 pounds and actual friction counter forces may be as high as 5 pounds from the foam/bed sheet interface resulting in completely dysfunctional and possible health threatening situations due to improper traction pull in relation to a defined physicians request.

(f) Although prior art designs have straps that wrap around a circumference, they do not maximize the strap compression surface area when they wrap at 90 degrees to the straight leg line (drawn from the heel to the ischial tuberosity). At only one increment, the proximal edge of the wrapped strap, does the compressive strap on current devices apply the constriction resulting in both inefficient compression and the danger of a tourniquet effect on the limb.

(g) Medical cases requiring the use of bucks traction boots are often pre and post surgical cases which would require the use of external intermittent pneumatic compression in order to prevent/provide prophylaxis for Deep Vein Thrombosis (venous thromboembolism). Because of the nature of the current designs the Bucks boot covers the foot and there is no provision for IPC devices to be used with the boots. This deprives the patient of critical lifesaving prophylaxis that is now a standard of care to combat Deep Vein Thrombosis and Pulmonary Embolism in addition to depriving the patient of natural circulation to the lower extremities.

(h) There is little care observed in the design of the foot pocket Prior art defines boots cut and sewn to basically a 90-degree angle on the foot portion of the boot in relation to the leg portion which makes up the foot pocket. There is no prior art which reflects the anatomically correct pocket for the heel as it lies on the bed. There are two dimensions which are important in the prevention of skin breakdown and in the proper positioning of the foot. The first is the back of the heel which extends beyond the line of the calf. The second is the base of the heel which extends beyond the fifth metatarsal. Both these extensions of dimension require a pocket to be designed in the device so as distribute the pressure from the bony prominence of the heel throughout the leg and support the foot (prevent it from dropping).

2. Objects and Advantages

Accordingly, besides the objects and advantages of the Best Bucks Multifunction Traction Boot, several objects and advantages of the present invention are:

(a) to provide a unique orthopedic traction boot for leg extension traction (Bucks) that, opposed to a symmetric cradle which is held tight to the leg by thin straps tightened 90 degrees to the line of the leg is asymmetrical in shape to allow for a tension wrapping of the leg from distal to proximal and wide straps that wrap in a spiral up the leg to maximize the surface area of the strap and due to their self limiting wrapping feature eliminate the possibility of over tightening.

(b) the use of a single circular ring that moves along a stirrup webbing that provides perfect center pull on the leg and through the elimination of friction eliminates any potential off center pulling due to leg movements.

(c) The universal size of the boot that enables it to fit variable circumferences of leg and additionally variable lengths of leg since the wrapping and strapping of the device provide the strength to hold the device on the leg.

(d) To provide an open foot dorsal surface on a device without the dorsal strap to facilitate neurological checks/palpation or observation of all the foot surfaces and eliminate the potential for neurological dysfunction from straps that could become over tightened

(e) To minimize the friction component of the leg weight on the foam, which rests on the bed, and is required to be pulled at a certain weight of pull for the traction to be effective

(f) To allow by design the ability for the boot to be used as a combination device with an external/intermittent pneumatic compression device that compresses the foot and thereby have the combination device together for use when the emergency arises.

(g) to allow by design a unique foot pocket that allows for space at the back and the bottom of the heel space and supports the heel so that there is no abnormal pressure on the heel with the patient lying on the back and the foot is not dropping forward.

### SUMMARY

In accordance with the present invention a universal multifunction bucks traction boot comprises a boot which is universal, asymmetrically shaped and designed to be wrapped around the leg and secured with straps in a distal to proximal wrap, secured by wide straps which prevent over-tightening, an open strapless area for observation and testing of the foot with an actual heel pocket and slight dorsiflexion rigidity, a free running ring in a stirrup for the actual traction tension application, with a more friction free surface than the standard foam that reduces the boot/bed friction interface and the space to position and include in the boot device an external/intermittent pneumatic compression device for the foot for use with the device when prevention of Deep Vein Thrombosis is an issue.

### DRAWINGS

Drawing figures

In the drawings, closely related figures have the same number but different alphabetic suffixes.

FIG. 1 shows the asymmetrical shape of the boot which allows its unique wrapping of the leg before the tension straps are applied and the special cut of the pattern that becomes the heel pocket and the open area to assess the dorsum of the foot.

FIG. 2 shows the wrap of the garment from distal to proximal and unique ring attachment that travels free and frictionless on the webbing.

FIG. 3 shows a top view of the second phase of the leg wrap before the tension straps are applied.

FIG. 4 shows a side view of the boot applied to reveal the cut outs and the lack of a dorsal strap in the area of the dorsum of the foot. This side view additionally shows the friction dissipating material under the device which minimizes the extra weight needed by the ortho technician to apply to over come the leg/bed interface.

FIG. 5 shows the open traction boot with the external/intermittent pneumatic compression garment laying inside the boot and its connection tubing outside to allow hook up with a pneumatic pump. The IPC garment can be positioned and or removed from inside the traction boot as its compression is against the plantar plexus which is accessible in the uniquely designed cutout.

Reference Numerals in Drawings

| | |
|---|---|
| 10 Anatomically correct | 12 Friction free ring attachment |
| asymmetrical shape | 16 Top view step 2 application |
| 14 Top view of boot in first | 20 Open boot with IPC device |
| application step | 24-Foot/heel pocket shape |
| 18 Side view of boot with | 28 Friction dissipation fabric |
| cutouts for foot | 32 Zipper or sewn boot end |
| 22 Wide straps in spiral wrap | 36 Straps |
| 26 Zipper access for IPC embodiment | |
| 30 Convoluted foam peaks | |
| 34 Pull webbing | |

### DETAILED DESCRIPTION

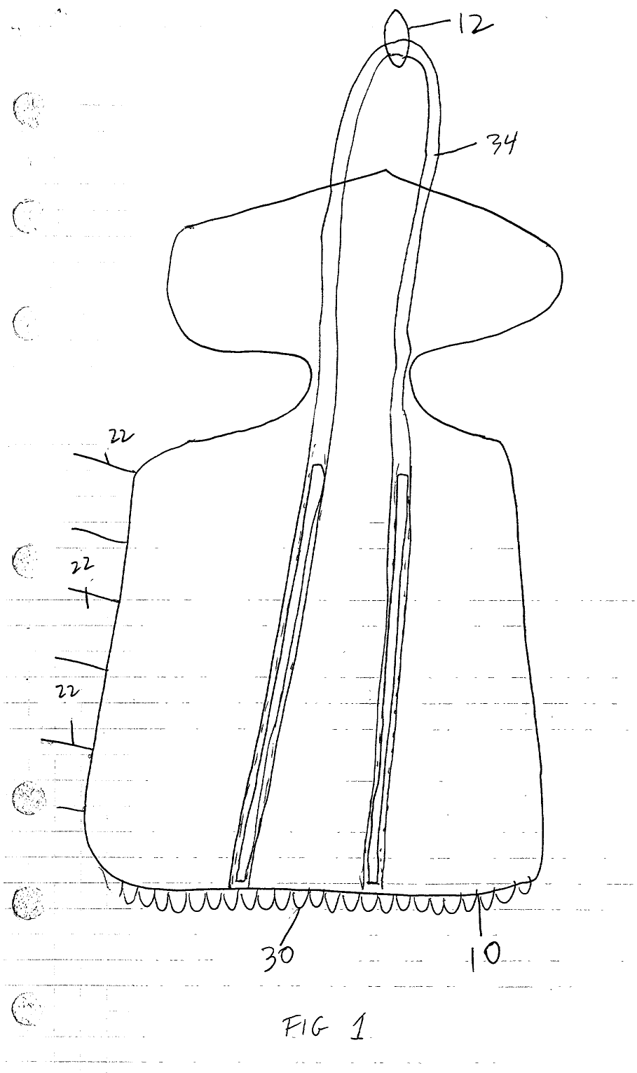

Description—FIG. 1

A preferred embodiment of the present invention is illustrated in FIG. 1. The unique shape is cut out of convoluted foam with the convolutions facing the skin. The opposite side of the convoluted material is the hook compatible or loop surface which covers the back. This material additionally causes far less friction than the foam by itself. Their 30 peaks actually become the tactile frictional component that is responsible for holding the device on the leg. Because of the unique wrapping of the device as opposed to the mere formation of a trough for the leg to rest in prior art means that a higher number of convoluted peaks will actually be contacting all surfaces of the leg. Also in FIG. 1 is the webbing and stays that allow the unique friction free ring to function for the centerline pull on the leg. The unique obtuse angle at the end of the boot with the friction free ring is the shape needed so when a 32 zipper or sewn end of the boot is completed, a unique 24 heel pocket is formed that supports the plantar surface and relieves pressure at the back of the heel. The extreme cutaway of the dorsal area is the feature that allows visual and neurological assessment of the foot. This is accomplished in this unique design because of the unique method of application which wraps the leg with the foam thereby reducing the need for the number of straps and a dorsal strap and hence a dorsal covering. Additionally, the straps can be seen to be sewn at a different angle to the centerline than the standard placement of 90 degrees to the centerline of the leg, resulting in a slight spiral wrap, following the wrap of the boot, using a lot less force but a much greater surface area of contact with the convoluted peaks of the foam. The foam in an addition to 30 convoluted peaks to remove the chances of vascular occlusion at the skin surface is also of a specific density. It is soft enough so as not to initiate any sort of maceration or skin breakdown but with a high enough density so that when used with standard hook and loop closures, if wrapped too tight the hook and loop closure will release to an appropriate tension on the device yet still hold it wrapped around the leg.

The 34-pull webbing is sewn along one side of a symmetrical pull line that will accommodate a range of calf and leg dimensions. A flexible plastic stay is trapped in the sewn webbing to keep the device rigid for application. Before the completion of the sewing the friction free ring is placed on the strap. The second side with stay trapped inside the webbing is sewn on the foam shape. The straps are placed so their application will follow 90 degrees to the direction of the wrap which will be discussed in the operation of the device. Three wide straps are sewn spaced apart on the available areas above the dorsal cutaway. The sides of the zipper are attached to the areas that will fold together to form the heel pocket. The zipper is then connected and zipped up to form the heel pocket. This is the area where the External/Intermittent pneumatic compression device is placed

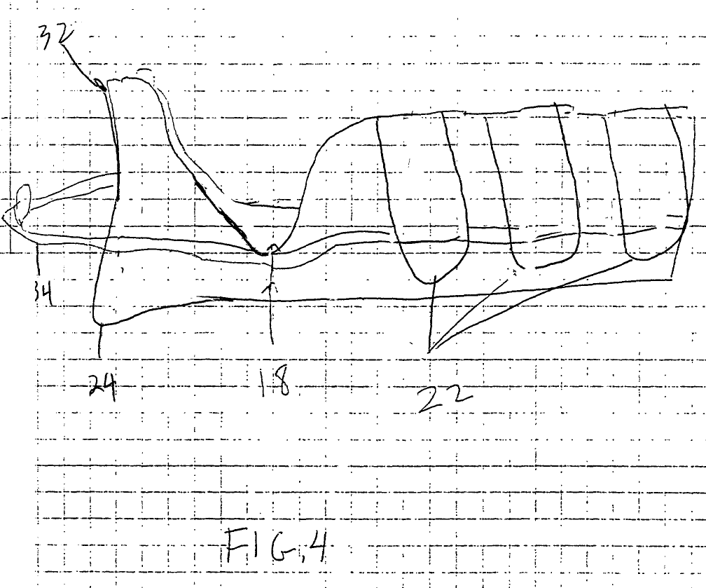

FIG. 4 Additional Embodiments

Additional embodiments are shown in FIG. 4. If a less costly version of the boot is desired, a hook compatible strip that provides for the attachment of the straps that runs from the dorsal cutaway to the end of the boot. Another part of the additional embodiment is the placement of a friction free material attached under the boot since in this embodiment the foam is not totally covered on the exterior surface with the hook compatible material.

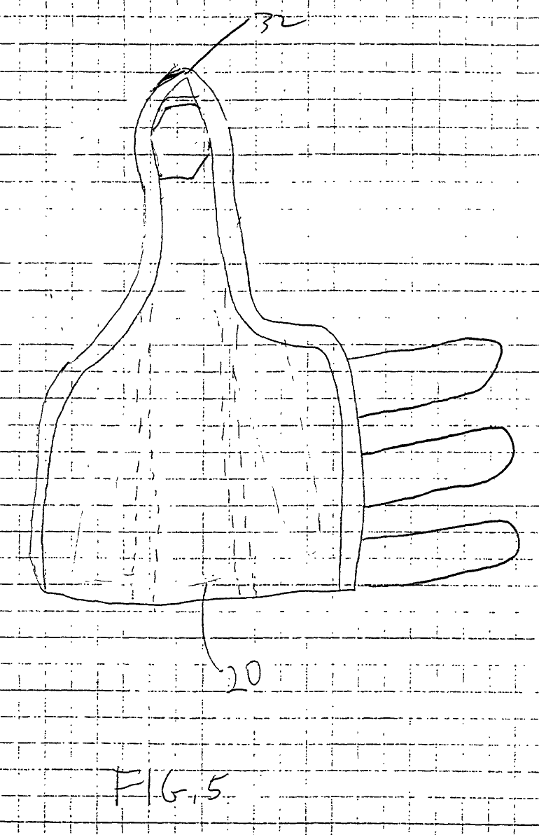

FIG. 5 Alternative Embodiments

Alternative embodiments are based on the drawing in FIG. 5. If the boot is not to be used as a combination device with the external/intermittent pneumatic compression, then the dorsal part with the heel pocket can be sewn or permanently attached as there is no need to have the ability to open the dorsal portion for the application of the external/intermittent pneumatic compression device for the foot. The dorsal cutaway of the boot is open sufficiently for access to all pedal pulse, neurological and visual assessments.

Advantages

From the description above, a number of advantages of the Best Multifunction Bucks traction boot become evident:

(a) The use of a universal boot over a sized boot saves on storage space and inventory costs, as there is only one size to carry.

(b) The Best Multifunction Bucks boot will eliminate problems from tension straps applied too tight since the asymmetrical shaped boot is wrapped around the leg from distal to proximal and the extra wide straps in their unique spacing forces them to be wrapped in a slight spiral fashion allowing their complete surface area along with the surface area of the wrapped convoluted foam to distribute the strapping tension around the leg. The balance of the foam density which resists an over tightened wrap and the yield point of the tensile strength of the loop compatible closures insures that the straps can not be over tightened.

(c) The use of a single circular ring that slides along the stirrup webbing attached to the boot insures that the pull on the boot is always down the centerline of the leg. The distribution of the pulling force has nothing to push it one way or another or cause it to fall off. The ring is an easy hitching area for the traction cord.

(d) The open strapless dorsal area of the foot eliminates the chance for any kind of inappropriate strap tension on the foot area and provides easy access for pedal pulse assessment, neurological checks, skin integrity and coloration assessment and even exercise. With the zipper feature in place there is easy access to the placement, treatment, or removal of the external/intermittent pneumatic compression device on the boot.

(e) The surface under the boot minimizes the friction against the bed interface and thereby provides a more realistic traction treatment without the need to add extra weight to overcome the frictional component of pulling on a leg wrapped in open cell foam resting on a bed surface.

(f) The combination device which includes the Best Multifunction Bucks traction boot and an external/intermittent pneumatic compression device for the prevention of Deep Vein Thrombosis allows the critical care device to be prepared for application at the time of the boot application for those cases indicating this type of treatment. With the lack of a need for a dorsal strap and the zipper feature and large dorsal cutout in the area of the foot allow these devices to be used in combination at the same time.

(g) The unique shape of the design before completion or attachment insures a unique heel pocket to be formed both in the back of the heel which prevents abnormal pressures from being placed on the bony prominence of the heel and at the base of the heel which allows for anatomically correct foot support that will prevent foot drop by placing the foot in a slightly dorsiflexed position.

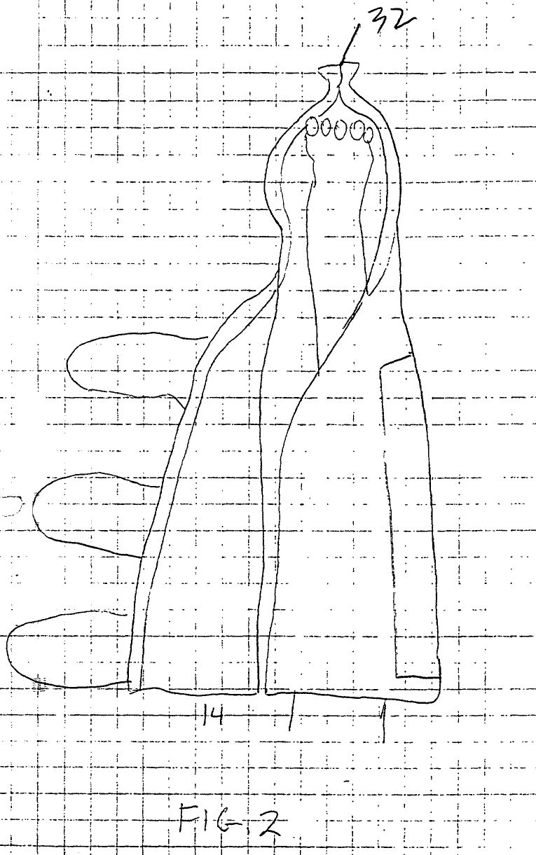

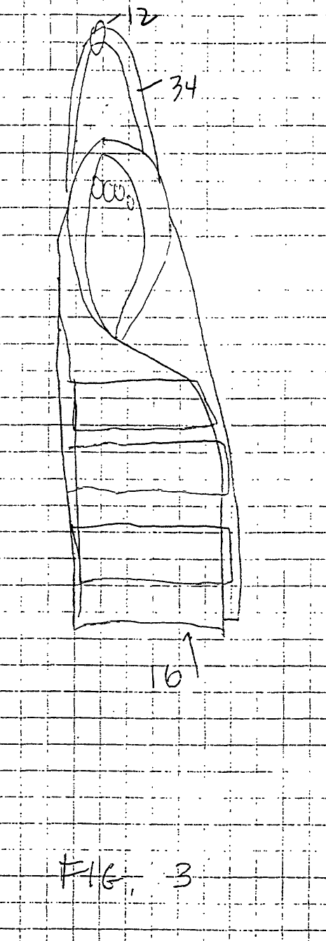

Operation—FIGS. 1, 2, 3, 4, 5

The manner of using the Best Multifunction Bucks traction boot is different from the boots in present day use. Namely, the portion of the boot above the ankle is actually wrapped in a compression wrap from the ankle proximally toward the knee. The first step is to place the patient's heel all the way into the boot. Once the lower leg is properly positioned in the boot, the sides of the boot are to be wrapped around the patient's leg in the following manner: First: Begin with the convoluted foam side with the Velcro straps attached. Wrap this side around the patient's lower leg. Second: Wrap the other foam side with the attachment strap snugly around and over the first foam wrap. The Velcro straps may now be tightened beginning with the most distal strap and working proximally up the lower leg. The straps will wrap 90 degrees to the increasing taper of the leg which will appear to be a slight spiral up the leg. This will maximize the effective surface area of the wrap yet not over tighten on either edge of the strap.

The straps should be snug enough to hold the boot properly in place during traction. However, the straps should not be too tight as to cause discomfort or pressure, especially if there is injury to the leg Strap tightness should be progressively less tight as you work proximally up the leg. When the wrapping is complete, the boot should be evenly and symmetrically distributed along the length of lower leg. Recheck for distal pulse, dorsi-flexion and plantar-flexion

Conclusion, Ramifications, and Scope

Accordingly, the reader will see the Best Multifunction Bucks boot will offer a multitude of special features as well as its unique special application for comfort and functionality. The design of the boot makes it a universal fit which will save on storage space and inventory costs, as there is only one size to carry. The design also will eliminate problems from the tension straps being applied too tight since the specialized wrapping allows the wide straps to distribute the strapping tension around the leg and prevent over tightening. This design incorporates the unique circular ring which slides on a stirrup made of webbing that is sewn to the boot and insures that the pull on the boot is always down the centerline of the leg. This is different than other boots on the market which utilize either a spreader plate or spreader bars which have inherent problems related to their design that are remedied by the free moving circular ring. The dorsal area of the foot is open for easy access for palpation, pedal pulse assessment, neurological checks or even exercise. It does not need a strap in this area like other designs, as the wrapping of the boot around the leg is secure for the heaviest of traction pulls. Furthermore, the Best Multifunction Bucks Boot has the additional advantages in that It permits the combination device of bucks boot with external/intermittent pneumatic compression through the use of a zipper designed at the base of the foot to facilitate treatments which require a bucks traction and the use of a pneumatic compression device for the prevention of DVT and PE;

It allows for a truer traction as the surface under the boot minimizes the friction against the bed interface and therefore does not require extra weight to overcome the frictional component of the boot/bed interface often found with foam boots;

It provides a unique shape for the heel pocket to be formed both in the back of the heel which prevents abnormal pressures from being placed on the bony prominence of the heel and at the base of the heel which allows for anatomically correct foot support that will prevent foot drop by placing the foot in a slightly dorsiflexed position.

Although the description above contains many specificities, these should not be construed as limiting the scope of the invention but as merely providing illustrations of some of the presently preferred embodiments of this invention. For example, the universal fit can be miniaturized for the pediatric patient, the friction free material on the bottom of the boot can be of several materials, etc.

Thus the scope of the invention should be determined by the appended claims and their legal equivalents, rather than by the examples given.

I claim:

1. In an orthopedic traction boot for bucks type traction wherein the improvement comprises the shape of the design and density of the material and its influence on its universal application.

2. The asymmetrical shape of the material in claim 1 permits the application to include compressive wrapping of one side of the boot around the leg from distal to proximal to be followed by the compressive wrap of the other side of the boot from distal to proximal for universal fitting.

3. The minimal number of wide straps are positioned on the shape in claim 1, so they wrap the compressive boot material ninety (90) degrees to the taper of the leg (surface)

4. The density of the boot material to make the shape in claim 1 which resists compression and the maximum hold strength of the straps on the hook/loop compatible material combines to form a strap closure system that resists over tightening.

5. The assembled shape of claim 1 creates an enlarged pocket for the heel in three dimensions.

6. The assembled shape of claim 1 creates an open cutout for examination of the dorsal area of the foot and an opening for the combining of the boot with the external/intermittent pneumatic compression device.

7. A webbing stirrup reinforced by two lateral stays with a free moving circular ring provides the attachment point for the traction.

8. Friction dissipative material on the lower surface that contacts the bed of the assembled claim 1 material allows for its friction reduction during use.
